SEQUENCE LISTING (19 sequences)

SEQ ID NO: 1 - DNA, 21 bp, Gibberella zeae
  misc_feature (1)..(2): n = a, t, c, g, or u
nncgucggaagagguaggaag

SEQ ID NO: 2 - DNA, 21 bp, Gibberella zeae
  misc_feature (20)..(21): n = a, t, c, g, or u
auuccuaccugauccgaggnn

SEQ ID NO: 3 - DNA, 21 bp, Gibberella zeae
  misc_feature (1)..(2): n = a, t, c, g, or u
nncggcugauguuaagcauag

SEQ ID NO: 4 - DNA, 21 bp, Gibberella zeae
  misc_feature (20)..(21): n = a, t, c, g, or u
auaugcuuaaguucagcggnn

SEQ ID NO: 5 - DNA, 21 bp, Gibberella zeae
  misc_feature (1)..(2): n = a, t, c, g, or u
nnauaucaauaagcggaggaa

SEQ ID NO: 6 - DNA, 21 bp, Gibberella zeae
  misc_feature (20)..(21): n = a, t, c, g, or u
uuccuccgcuuauugauaunn

SEQ ID NO: 7 - DNA, 21 bp, Gibberella zeae
  misc_feature (1)..(2): n = a, t, c, g, or u
nnccuaguaacggcgagugaa

SEQ ID NO: 8 - DNA, 21 bp, Gibberella zeae
  misc_feature (20)..(21): n = a, t, c, g, or u
uucacucgccguuacuaggnn

SEQ ID NO: 9 - DNA, 21 bp, Artificial Sequence
  nonsense sequence
  misc_feature (1)..(2): n = a, t, c, g, or u
nnuacgacgaggcacuagagu

SEQ ID NO: 10 - DNA, 21 bp, Artificial Sequence
  nonsense sequence
  misc_feature (20)..(21): n = a, t, c, g, or u
acucuagugccucgucguann

SEQ ID NO: 11 - DNA, 661 bp, Gibberella zeae
  rRNA (1)..(661): DNA encoding 28S ribosomal RNA
  ref <308>: DATABASE ACCESSION NUMBER: AY188924
  ref <309>: DATABASE ENTRY DATE: 2005-11-08
  ref <313>: RELEVANT RESIDUES IN SEQ ID NO: (1)..(661)
aacttctgaatgttgacctcggatcaggtaggaatacccgctgaacttaagcatatcaataagcggaggaaaagaaaccaacagggattgccctagtaacggcgagtgaagcggcaacagctcaaatttgaaatctggcttttcgggcccgagttgtaatttgtagaggatgattttgatgcggtgccttccgagttccctggaacgggacgccatagagggtgagagccccgtctggttggatgccaaatctctgtaaatctccttcgacgagtcgagtagtttgggaatgctgctctaaatgggaggtatatgtcttctaaagctaaataccggccagagaccgatagcgcacaagtagagtgatcgaaagatgaaaagcactttgaaaagagagttaaaaagtacgtgaaattgttgaaagggaagcgtttatgaccagacttgggcttggttaatcatctggggttctctccagtgcacttttccagtccaggccagcatcagttttcgccgggggataaaggcttcgggaatgtggctcccctcggggagtgttatagcccgttgtgtaataccctggtggggactgaggttcgcgcttctgcaaggatgctggcgtaatggtcagcaacgacccgtcttgaaacacggaccaagg

SEQ ID NO: 12 - RNA, 207 nt, Glycine max
  ref <301>: AUTHORS: Subramanian S, Fu Y, Sunkar R, Barbazuk WB, Zhu JK, Yu O
  ref <302>: TITLE: Novel and nodulatio-regulated microRNAs in soybean roots
  ref <303>: JOURNAL: BMC Genomics
  ref <304>: VOLUME: 9
  ref <306>: PAGES: 160
  ref <307>: DATE: 2008-04-10
  ref <313>: RELEVANT RESIDUES IN SEQ ID NO: (1)..(207)
cguugaagacccuaagguaagagagcuuucuucaguccacucaugggugacaguaagauucaauuagcugccgacucauucauccaaauguugaguguaagcgaauaaauauacucagcagaugagugaaugaugcgggagacaaauugaaucuuaaguuuccuguacuuggacugaagggagcucccuuuuccuuuugucucuuac

SEQ ID NO: 13 - DNA, 439 bp, Artificial Sequence
  artificial sequence
tcccgttgaagaccctaaggtaaacggctgatgttaagcatagtcatgggtgacagtaagattcaattagctgccgactcattcatccaaatgttgagtgtaagcgaataaatatactcagcagatgagtgaatgatgcgggagacaaattgaatcttaagtttcctgtacatatgcttaagttcagcggtttttccttttgtctcttacagatcccgttgaagaccctaaggtaaacgtcggaagaggtaggaagtcatgggtgacagtaagattcaattagctgccgactcattcatccaaatgttgagtgtaagcgaataaatatactcagcagatgagtgaatgatgcgggagacaaattgaatcttaagtttcctgtacattcctacctgatccgaggtttttccttttgtctcttacgagctcttcatatgac

SEQ ID NO: 14 - DNA, 9111 bp, Artificial Sequence
  binary vector 18911
attcctgtggttggcatgcacatacaaatggacgaacggataaaccttttcacgcccttttaaatatccgattattctaataaacgctcttttctcttaggtttacccgccaatatatcctgtcaaacactgatagtttaaacgggaccctcatgagcggagaattaagggagtcacgttatgacccccgccgatgacgcgggacaagccgttttacgtttggaactgacagaaccgcaacgaagctttggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtggatcccgttgaagaccctaaggtaaacggctgatgttaagcatagtcatgggtgacagtaagattcaattagctgccgactcattcatccaaatgttgagtgtaagcgaataaatatactcagcagatgagtgaatgatgcgggagacaaattgaatcttaagtttcctgtacatatgcttaagttcagcggtttttccttttgtctcttacagatcccgttgaagaccctaaggtaaacgtcggaagaggtaggaagtcatgggtgacagtaagattcaattagctgccgactcattcatccaaatgttgagtgtaagcgaataaatatactcagcagatgagtgaatgatgcgggagacaaattgaatcttaagtttcctgtacattcctacctgatccgaggtttttccttttgtctcttacgagctcttcatatgacgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgcggacccagctgcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaactcgagacttttcaacaaagggtaatatccggaaacctcctcggattccattgcccagctatctgtcactttattgtgaagatagtggaaaaggaaggtggctcctacaaatgccatcattgcgataaaggaaaggctatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgaacaatcccactatccttctgccggaccctcatgagcggagaattaagggagtcacgttatgacccccgccgatgacgcgggacaagccgttttacgtttggaactgacagaaccgcaacgaagctttggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttggcatatgtaaatcactgccatcacgcggatcactaatatggaaccgtcgattaaaacagatcgacggtttatacatcattttattgtacacacggatcgatatctcagccgttagatttaatatgcgatctcatatgtccactcacccgtcagtctataaatacttagcccctccctcattgttaagggagcaaaatctcagagagatagtcctagagagagaaagagagcaagtagcctagaagtggatcctaaaccatgaagaagccagaacttaccgctacttccgttgagaagttcctcattgagaagttcgattccgtgtccgatcttatgcaactttctgaaggtgaagagtctagggctttctctttcgatgttggtggaaggggatacgttttgagagttaactcttgcgctgacggcttctacaaggatagatacgtttacaggcacttcgcttcagctgctttgccaattccagaggttttggatattggagagttctctgagtccctcacctattgcatttctagaagggctcaaggtgtgactcttcaagatcttccagaaactgagcttccagctgttcttcaaccagttgctgaagctatggatgctattgctgctgctgatctttctcaaacttctggattcggaccattcggtccacaaggtattggacagtacactacttggagggatttcatttgcgctatcgctgatccacatgtttaccattggcagaccgttatggatgataccgtttctgcttctgttgctcaagctcttgatgagcttatgctttgggctgaagattgcccagaggttagacatcttgttcatgctgatttcggctctaacaacgtgttgactgataacggaaggattaccgctgtgattgattggtctgaagctatgttcggagactctcaatacgaggtggccaacatattcttttggaggccttggcttgcttgcatggaacaacaaactagatacttcgagagaaggcatccagaacttgctggatctccaagacttagagcttacatgcttaggattggactcgatcagctttaccagtctcttgttgatggcaacttcgatgatgctgcttgggctcagggaagatgtgatgctattgtgagatctggtgctggaactgttggaagaactcaaattgctagaaggtctgctgctgtttggactgatggatgcgttgaagttcttgctgattctggaaacagaaggccatctactagaccaagggctaaagagtgagagctcgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctgctagccctgcaggaaatttaccggtgcccgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccagggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctggctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 15 - DNA, 10373 bp, Artificial Sequence
  binary vector 18624
attcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgctgcaggaattggccgcagcggccatttaaatcaattgggcgcgccgaattcgagctcggtacaagcttgggaccccggaccctcatgagcggagaattaagggagtcacgttatgacccccgccgatgacgcgggacaagccgttttacgtttggaactgacagaaccgcaacgaagctttggcagacaaagtggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtggatcccgttgaagaccctaaggtaaacggctgatgttaagcatagtcatgggtgacagtaagattcaattagctgccgactcattcatccaaatgttgagtgtaagcgaataaatatactcagcagatgagtgaatgatgcgggagacaaattgaatcttaagtttcctgtacatatgcttaagttcagcggtttttccttttgtctcttacagatcccgttgaagaccctaaggtaaacgtcggaagaggtaggaagtcatgggtgacagtaagattcaattagctgccgactcattcatccaaatgttgagtgtaagcgaataaatatactcagcagatgagtgaatgatgcgggagacaaattgaatcttaagtttcctgtacattcctacctgatccgaggtttttccttttgtctcttacgagctcttcatatgacgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgcggaccgggtaccagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagggatccccgatcatgcaaaaactcattaactcagtgcaaaactatgcctggggcagcaaaacggcgttgactgaactttatggtatggaaaatccgtccagccagccgatggccgagctgtggatgggcgcacatccgaaaagcagttcacgagtgcagaatgccgccggagatatcgtttcactgcgtgatgtgattgagagtgataaatcgactctgctcggagaggccgttgccaaacgctttggcgaactgcctttcctgttcaaagtattatgcgcagcacagccactctccattcaggttcatccaaacaaacacaattctgaaatcggttttgccaaagaaaatgccgcaggtatcccgatggatgccgccgagcgtaactataaagatcctaaccacaagccggagctggtttttgcgctgacgcctttccttgcgatgaacgcgtttcgtgaattttccgagattgtctccctactccagccggtcgcaggtgcacatccggcgattgctcactttttacaacagcctgatgccgaacgtttaagcgaactgttcgccagcctgttgaatatgcagggtgaagaaaaatcccgcgcgctggcgattttaaaatcggccctcgatagccagcagggtgaaccgtggcaaacgattcgtttaatttctgaattttacccggaagacagcggtctgttctccccgctattgctgaatgtggtgaaattgaaccctggcgaagcgatgttcctgttcgctgaaacaccgcacgcttacctgcaaggcgtggcgctggaagtgatggcaaactccgataacgtgctgcgtgcgggtctgacgcctaaatacattgatattccggaactggttgccaatgtgaaattcgaagccaaaccggctaaccagttgttgacccagccggtgaaacaaggtgcagaactggacttcccgattccagtggatgattttgccttctcgctgcatgaccttagtgataaagaaaccaccattagccagcagagtgccgccattttgttctgcgtcgaaggcgatgcaacgttgtggaaaggttctcagcagttacagcttaaaccgggtgaatcagcgtttattgccgccaacgaatcaccggtgactgtcaaaggccacggccgtttagcgcgtgtttacaacaagctgtaagagcttactgaaaaaattaacatctcttgctaagctgggagctcgatccgtcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctgctagccctgcaggaaatttaccggtgcccgggcggccagcatggccgtatccgcaatgtgttattaagttgtctaagcgtcaatttgtttacaccacaatatatcctgccaccagccagccaacagctccccgaccggcagctcggcacaaaatcaccactcgatacaggcagcccatcagaattaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcacacaggaaacagaccatgagggaagcgttgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaagtagtcggcaaataaagctctagtggatctccgtacccggggatctggctcgcggcggacgcacgacgccggggcgagaccataggcgatctcctaaatcaatagtagctgtaacctcgaagcgtttcacttgtaacaacgattgagaatttttgtcataaaattgaaatacttggttcgcatttttgtcatccgcggtcagccgcaattctgacgaactgcccatttagctggagatgattgtacatccttcacgtgaaaatttctcaagcgctgtgaacaagggttcagattttagattgaaaggtgagccgttgaaacacgttcttcttgtcgatgacgacgtcgctatgcggcatcttattattgaataccttacgatccacgccttcaaagtgaccgcggtagccgacagcacccagttcacaagagtactctcttccgcgacggtcgatgtcgtggttgttgatctagatttaggtcgtgaagatgggctcgagatcgttcgtaatctggcggcaaagtctgatattccaatcataattatcagtggcgaccgccttgaggagacggataaagttgttgcactcgagctaggagcaagtgattttatcgctaagccgttcagtatcagagagtttctagcacgcattcgggttgccttgcgcgtgcgccccaacgttgtccgctccaaagaccgacggtctttttgttttactgactggacacttaatctcaggcaacgtcgcttgatgtccgaagctggcggtgaggtgaaacttacggcaggtgagttcaatcttctcctcgcgtttttagagaaaccccgcgacgttctatcgcgcgagcaacttctcattgccagtcgagtacgcgacgaggaggtttatgacaggagtatagatgttctcattttgaggctgcgccgcaaacttgaggcagatccgtcaagccctcaactgataaaaacagcaagaggtgccggttatttctttgacgcggacgtgcaggtttcgcacggggggacgatggcagcctgagccaattcccagatccccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagctctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatccggaatta

SEQ ID NO: 16 - RNA, 207 nt, Artificial Sequence
  FgRNA-1 miRNA stem-loop
cguugaagacccuaagguaaacgucggaagagguaggaagucaugggugacaguaagauucaauuagcugccgacucauucauccaaauguugaguguaagcgaauaaauauacucagcagaugagugaaugaugcgggagacaaauugaaucuuaaguuuccuguacauuccuaccugauccgagguuuuuccuuuugucucuuac

SEQ ID NO: 17 - RNA, 207 nt, Artificial Sequence
  FgRNA-2 miRNA stem-loop
cguugaagacccuaagguaaacggcugauguuaagcauagucaugggugacaguaagauucaauuagcugccgacucauucauccaaauguugaguguaagcgaauaaauauacucagcagaugagugaaugaugcgggagacaaauugaaucuuaaguuuccuguacauaugcuuaaguucagcgguuuuuccuuuugucucuuac

SEQ ID NO: 18 - RNA, 21 nt, Gibberella zeae
  misc_feature (1)..(2): n = a, t, c, g, or u
nnccucggaucagguaggaau

SEQ ID NO: 19 - RNA, 21 nt, Gibberella zeae
  misc_feature (1)..(2): n = a, t, c, g, or u
nnccgcugaacuuaagcauau

(19) United States
(12) Patent Application Publication
(10) Pub. No.: US 2014/0259222 A1
(43) Pub. Date: Sep. 11, 2014

(54) PATHOGEN RESISTANCE

(71) Applicant: Syngenta Participations AG, Basel (CH)

(72) Inventors: Xiang Huang, Research Triangle Park, NC (US); John Daniel Hipskind, Research Triangle Park, NC (US)

(73) Assignee: Syngenta Participation AG, Basel (CH)

(21) Appl. No.: 14/345,969

(22) PCT Filed: Sep. 20, 2012
(86) PCT No.: PCT/US2012/056184

§ 371 Date: Mar. 20, 2014

Related U.S. Application Data
(60) Provisional application No. 61/537,195, filed on Sep. 21, 2011.

(51) Int. Cl. C12N 15/82; A01H 5/00

(52) U.S. Cl. CPC C12N 15/8282; A01H 5/00
USPC 800/279; 435/419; 435/320.1; 536/23.74; 800/301

(57) ABSTRACT

Disease in food crops caused by fungal pathogens is a major concern to the agricultural industry, with annual losses typically in the billions of dollars. Fusarium graminearum, also known as Gibberella zeae, is known to cause, among other diseases, headblight disease in wheat and stalk and ear rot in maize. Disease caused by Fusarium graminearum has proven to be a difficult disease to manage because of limitations of control options. Disclosed herein are nucleic acid sequences which have been proven to provide corn and soybean with resistance to Fusarium graminearum. Also disclosed herein are methods of using the nucleic acid sequences, and plants comprising the nucleic acid sequences.

### FIELD OF THE INVENTION

The present invention relates generally to the field of plant molecular biology. More specifically, the present invention relates to methods and compositions for fungal pathogen control in plants. More particularly, it discloses transgenic plant cells, plants and seeds comprising recombinant DNA and methods of making and using such plant cells, plants and seeds that are associated with fungal pathogen resistance.

### BACKGROUND

Disease in food crops caused by fungal pathogens is a major concern to the agricultural industry, with annual losses typically in the billions of dollars. The genus Fusarium collectively represents the most important group of fungal plant pathogens, causing various diseases on nearly every economically important plant species. Fusarium graminearum, also known as Gibberella zeae, is known to cause, among other diseases, headblight disease in wheat and stalk and ear rot in maize. During the springs of 2004 and 2005, 112 isolates of Fusarium graminearum were recovered from diseased corn and soybean seedlings from 30 locations in 13 Ohio counties. (Broders, K. D., Lipps, P. E., Paul, P. A., Dorrance, A. E. 2007. Plant Disease. 91(9):1155-1160). Estimated losses caused by headblight to growers, grain handlers, and industries that utilize wheat-related products in North Dakota, South Dakota, and Minnesota during 1993 alone exceeded $1 billion (McMullen, et al. 1997. Scab of wheat and barley: A re-emerging disease of devastating impact. Plant Dis. 81:1340-1348).

Fusarium graminearum can cause additional loss for agriculture because of the potent mycotoxins produced by the fungus. These mycotoxins have been tentatively linked with livestock toxicoses or feed refusal. Grain contaminated with Fusarium mycotoxins may be graded down or rejected entirely in commerce (Tuite, J., Shaner, G., and Everson, R. J. 1990. Wheat scab in soft red winter wheat in Indiana in 1986 and its relation to some quality measurements. Plant Dis. 74:959-962).

Disease caused by Fusarium graminearum has proven to be a difficult disease to manage because of limitations of control options. Infection is associated with rainfall during the flowering stage. The infection is spread by wind, birds, and planting infected seed. It should also be noted that the disease could survive on old crop residue for many years (Canadian Seed Trade Association, Fusarium graminearum—The Corn Seed Perspective, March 2003). Fungicide treatments have shown to be somewhat effective, however, costs of treatment and the difficulty of determining the optimum time of application make using fungicides less attractive to farmers (Bai and Shaner. 1994. Scab of wheat prospects for control. Plant Dis. 78:760-766; Martin, R. A., and Johnston, H. W. 1982. Effects and control of Fusarium diseases of cereal grains in the Atlantic Provinces. Can. J, Plant. Pathol. 4:210-216).

MicroRNAs (miRNAs) are non-protein coding RNAs, generally of between about 19 to about 25 nucleotides (commonly about 20-24 nucleotides in plants), that guide cleavage in trans of target transcripts, negatively regulating the expression of genes involved in various regulation and development pathways (Bartel (2004) Cell, 116:281-297). In some cases, miRNAs serve to guide in-phase processing of siRNA primary transcripts (see Allen et al. (2005) Cell, 121:207-221, which is incorporated herein by reference).

Some microRNA genes (MIR genes) have been identified and made publicly available in a database (‘miRBase”, available on line at microrna.sanger.ac.uk/sequences). Additional MIR genes and mature miRNAs are also described in U.S. Patent Application Publications 2005/0120415 and 2005/144669A1, which is incorporated by reference herein. MIR genes have been reported to occur in inter-genic regions, both isolated and in clusters in the genome, but can also be located entirely or partially within introns of other genes (both protein-coding and non-protein-coding). For a recent review of miRNA biogenesis, see Kim (2005) Nature Rev. Mol. Cell Biol., 6:376-385. Transcription of MIR genes can be, at least in some cases, under promotional control of a MIR gene's own promoter. MIR gene transcription is probably generally mediated by RNA polymerase II (see, e.g., Aukerman. and Sakai (2003) Plant Cell, 15:2730-2741; Parizotto et al. (2004) Genes Dev., 18:2237-2242), and therefore could be amenable to gene silencing approaches that have been used in other polymerase II-transcribed genes. The primary transcript (which can be polycistronic) termed a “pri-miRNA”, a miRNA precursor molecule that can be quite large (several kilobases) and contains one or more local double-stranded or “hairpin” regions as well as the usual 5′ “cap” and polyadenylated tail of an mRNA. See, for example, FIG. 1 in Kim (2005) Nature Rev. Mol. Cell Biol., 6:376-385.

In animal cells, this pri-miRNA is believed to be “cropped” by the nuclear RNase III Drosha to produce a shorter miRNA precursor molecule known as a “pre-miRNA”. Following nuclear processing by Drosha, pre-miRNAs are exported to the nucleus where the enzyme Dicer generates the short, mature miRNAs. See, for example, Lee et al. (2002) EMBO Journal, 21:4663-4670; Reinhart et al. (2002) Genes & Dev., 16:1616-1626; Lund et al. (2004) Science, 303:95-98; and Millar and Waterhouse (2005) Funct. Integr Genomics, 5:129-135, which are incorporated by reference herein. In contrast, in plant cells, microRNA precursor molecules are believed to be largely processed in the nucleus. Whereas in animals both miRNAs and siRNAs are believed to result from activity of the same DICER enzyme, in plants miRNAs and siRNAs are formed by distinct DICER-like (DCL) enzymes, and in Arabidopsis a nuclear DCL enzyme is believed to be required for mature miRNA formation (Xie et al. (2004) PLoS Biol., 2:642-652, which is incorporated by reference herein). Additional reviews on microRNA biogenesis and function are found, for example, in Bartel (2004) Cell, 116:281-297; Murchison and Hannon (2004) Curr. Opin. Cell Biol., 16:223-229; and Dugas and Bartel (2004) Curr. Opin. Plant Biol., 7:512-520. MicroRNAs can thus be described in terms of RNA (e.g., RNA sequence of a mature miRNA or a miRNA precursor RNA molecule), or in terms of DNA (e.g., DNA sequence corresponding to a mature miRNA RNA sequence or DNA sequence encoding a MIR gene or fragment of a MIR gene or a miRNA precursor).

MIR gene families appear to be substantial, estimated to account for 1% of at least some genomes and capable of influencing or regulating expression of about a third of all genes (see, for example, Tomari et al. (2005) Curr. Biol., 15:R61-64; G. Tang (2005) Trends Biochem. Sci., 30:106-14; Kim Nature Rev. Mol. Cell Biol., 6:376-385). Because miRNAs are important regulatory elements in eukaryotes, including animals and plants, transgenic suppression of miRNAs could, for example, lead to the understanding of important biological processes or allow the manipulation of certain pathways useful, for example, in biotechnological applications. For example, miRNAs are involved in regulation of cellular differentiation, proliferation and apoptosis, and are probably involved in the pathology of at least some diseases, including cancer, where miRNAs may function variously as oncogenes or as tumor suppressors. See, for example, O'Donnell et al. (2005) Nature, 435:839-843; Cai et al. (2005) Proc. Natl. Acad. Sci. USA, 102:5570-5575; Morris and McManus (2005) Sci. STKE, pe41 (available online at stke.sciencemag.org/cgi/reprint/sigtrans; 2005/297/pe41.pdf). MicroRNA (MIR) genes have identifying characteristics, including conservation among plant species, a stable foldback structure, and processing of a specific miRNA/miRNA* duplex by Dicer-like enzymes (Ambros et al. (2003) RNA, 9:277-279). These characteristics have been used to identify miRNAs and their corresponding genes in plants (Xie et al. (2005) Plant Physiol., 138:2145-2154; Jones-Rhoades and Bartel (2004) Mol. Cell, 14:787-799; Reinhart et al. (2002) Genes Dev., 16:1616-1626; Sunkar and Zhu (2004) Plant Cell, 16:2001-2019). Publicly available microRNA genes are catalogued at miRBase (Griffiths-Jones et al. (2003) Nucleic Acids Res., 31:439-441).

MiRNAs have been found to be expressed in very specific cell types in Arabidopsis (see, for example, Kidner and Martienssen (2004) Nature, 428:81-84, Millar and Gubler (2005) Plant Cell, 17:705-721). Suppression can be limited to a side, edge, or other division between cell types, and is believed to be required for proper cell type patterning and specification (see, for example, Palatnik et al. (2003) Nature, 425:257-263). Suppression of a GFP reporter gene containing an endogenous miR171 recognition site was found to limit expression to specific cells in transgenic Arabidopsis (Parizotto et al. (2004) Genes Dev., 18:2237-2242). Recognition sites of miRNAs have been validated in all regions of an mRNA, including the 5′ untranslated region, coding region, and 3′ untranslated region, indicating that the position of the miRNA target site relative to the coding sequence may not necessarily affect suppression (see, for example, Jones-Rhoades and Bartel (2004). Mol. Cell, 14:787-799, Rhoades et al. (2002) Cell, 110:513-520, Allen et al. (2004) Nat. Genet., 36:1282-1290, Sunkar and Zhu (2004) Plant Cell, 16:2001-2019).

For the forgoing reasons, there exists a need for an improved and reliable method of Fusarium graminearum control. The invention provides a solution to the problem identified using an engineered miRNA from soybean to comprise sequences effective at reducing the level of Fusarium graminearum disease in soybeans as well as in maize.

### SUMMARY

In one aspect, the present invention comprises a single-stranded nucleic acid molecule, or an isolated single-stranded nucleic acid molecule, comprising a first sequence and a second sequence, wherein the first sequence comprises a sequence obtained from a gene that encodes a fungal ribosomal RNA, and the second sequence comprises a sequence capable of forming a duplex with the first sequence. In another aspect, the fungal ribosomal RNA is from a fungus in the genus Fusarium. In another aspect, the fungal ribosomal RNA is the 28S ribosome from Fusarium graminearum. In another aspect, the first sequence is selected from the group consisting of SEQ ID NOs: 1, 3, 5, and 7. In another aspect, the second sequence is selected from the group consisting of SEQ ID NOs: 2, 4, 6, and 8. In another aspect, the single-stranded nucleic acid molecule further comprises a backbone sequence between the first sequence and the second sequence. In another aspect, the backbone sequence comprises at least nucleotides 41 to 167 of SEQ ID NO: 12. In another aspect, the single-stranded nucleic acid sequence is capable of forming a hairpin. In another aspect, the single-stranded nucleic acid molecule is synthetic. In another aspect, the nucleic acid is RNA or DNA or a DNA/RNA hybrid. In another aspect the single-stranded nucleic acid molecule is active against a Fusarium fungus or a Phakopsora fungus.

In another aspect, the present invention comprises an expression cassette comprising at least a first nucleic acid sequence which encodes for a first single-stranded nucleic acid molecule comprising a first sequence and a second sequence, wherein the first sequence comprises a sequence obtained from a gene that encodes a fungal ribosomal RNA, and the second sequence comprises a sequence capable of forming a duplex with the first sequence. In another aspect, the expression cassette further comprises a second nucleic acid sequence, wherein the first single-stranded molecule and the second single-stranded molecule do not comprise identical first sequences. In another aspect, the first single-stranded molecule comprises a first sequence selected from the group consisting of SEQ ID NOs: 1, 3, 5, and 7, and the second single-stranded molecule comprises a first sequence different from the first sequence in the first single-stranded molecule. In another aspect, the first single-stranded molecule comprises a first sequence comprising SEQ ID NO: 1, and the second single-stranded molecule comprises a first sequence comprising SEQ ID NO: 3. In another aspect, the expression cassette comprises SEQ ID NO: 13.

In another aspect, the present invention comprises a vector comprising an expression cassette comprising at least a first nucleic acid sequence which encodes for a first single-stranded nucleic acid molecule comprising a first sequence and a second sequence, wherein the first sequence comprises a sequence obtained from a gene that encodes a fungal ribosomal RNA, and the second sequence comprises a sequence capable of forming a duplex with the first sequence. In another aspect, the vector comprises SEQ ID NO: 14 or 15.

In another aspect, the present invention comprises a non-human host cell comprising an expression cassette comprising at least a first nucleic acid sequence which encodes for a first single-stranded nucleic acid molecule comprising a first sequence and a second sequence, wherein the first sequence comprises a sequence obtained from a gene that encodes a fungal ribosomal RNA, and the second sequence comprises a sequence capable of forming a duplex with the first sequence. In another aspect, the non-human host cell is selected from the group consisting of bacteria, virus, fungus, plant, and animal cells. In another aspect, the non-human host cell is a plant cell.

In another aspect, the present invention comprises a plant comprising a plant cell comprising an expression cassette comprising at least a first nucleic acid sequence which encodes for a first single-stranded nucleic acid molecule comprising a first sequence and a second sequence, wherein the first sequence comprises a sequence obtained from a gene that encodes a fungal ribosomal RNA and the second sequence comprises a sequence capable of forming a duplex with the first sequence. In another aspect, the plant is a monocot. In another aspect, the monocot is maize. In another aspect, the plant is a dicot. In another aspect, the dicot is soybean.

In another aspect, the present invention comprises a method of producing a plant resistant to a fungal pathogen, comprising the steps of: (a) obtaining an expression cassette comprising a nucleotide sequence encoding a single-stranded nucleic acid molecule, or an isolated single-stranded nucleic acid molecule, comprising a first sequence and a second sequence, wherein the first sequence comprises a sequence obtained from a gene that encodes a fungal ribosomal RNA, and the second sequence comprises a sequence capable of forming a duplex with the first sequence; (b) inserting the expression cassette into the genome of a plant cell; and (c) generating a plant from the plant cell; wherein the plant is resistant to a fungal pathogen. In another aspect, the isolated single-stranded nucleic acid molecule comprises a first sequence selected from the group consisting of SEQ ID NOs: 1, 3, 5, and 7. In another aspect, the plant cell is a maize plant cell. In another aspect, the plant is a maize plant. In another aspect, the plant cell is a soybean plant cell. In another aspect, the plant is a soybean plant. In another aspect, the method of the invention produces a plant that is resistant to a Fusarium fungus or a Phakopsora fungus.

### BRIEF DESCRIPTION OF THE SEQUENCES IN THE SEQUENCE LISTING

SEQ ID NO: 1 is the engineered FgRNA-1 passenger miRNA sequence.

SEQ ID NO: 2 is the FgRNA-1 guide antisense miRNA sequence.

SEQ ID NO: 3 is the engineered FgRNA-2 passenger miRNA sequence.

SEQ ID NO: 4 is the FgRNA-2 guide antisense miRNA sequence.

SEQ ID NO: 5 is the FgRNA-3 passenger miRNA sequence.

SEQ ID NO: 6 is the FgRNA-3 guide antisense miRNA sequence.

SEQ ID NO: 7 is the FgRNA-4 passenger miRNA sequence.

SEQ ID NO: 8 is the FgRNA-4 guide antisense miRNA sequence.

SEQ ID NO: 9 is the FgRNA-5 passenger miRNA nonsense sequence.

SEQ ID NO: 10 is the FgRNA-5 guide antisense miRNA nonsense sequence.

SEQ ID NO: 11 is a nucleotide sequence encoding a 28S ribosomal RNA from Fusarium graminearum.

SEQ ID NO: 12 is the endogenous soybean micro-RNA miR319 precursor.

SEQ ID NO: 13 is the cassette encoding FgRNA-1 and FgRNA-2 miRNA loops.

SEQ ID NO: 14 is soybean binary vector 18911.

SEQ ID NO: 15 is maize binary vector 18624.

SEQ ID NO: 16 is the FgRNA-1 miRNA stem-loop comprising passenger sequence SEQ ID NO: 1 and guide sequence SEQ ID NO: 2.

SEQ ID NO: 17 is the FgRNA-2 miRNA stem-loop comprising passenger sequence SEQ ID NO: 3 and guide sequence SEQ ID NO: 4.

SEQ ID NO: 18 is the FgRNA-1 passenger miRNA sequence prior to engineering.

SEQ ID NO: 19 is the FgRNA-2 passenger miRNA sequence prior to engineering

### DEFINITIONS

“Antisense inhibition” refers to the production of antisense RNA transcripts capable of suppressing the expression of protein from an endogenous gene or a transgene.

A “chimeric plant”, as used herein, refers to transformed plants that comprise non-transformed cells such that their specific transformed genotype will not be transferred sexually into the next generation. As a result, chimeric plants cannot be used in breeding techniques such as self-pollination.

A “chimeric sequence” is used to indicate a nucleic acid sequence, such as a vector or a gene, which is comprised of two or more nucleic acid sequences of distinct origin that are fused together, resulting in a nucleic acid sequence which does not occur naturally.

“Chromosomally-integrated” refers to the integration of a foreign gene or DNA construct into the host DNA by covalent bonds. Where genes are not “chromosomally integrated” they may be “transiently expressed.” Transient expression of a gene refers to the expression of a gene that is not integrated into the host chromosome but functions independently, either as part of an autonomously replicating plasmid or expression cassette, for example, or as part of another biological system such as a virus.

“Coding sequence” refers to a DNA or RNA sequence that codes for a specific amino acid sequence and excludes the non-coding sequences. It may constitute an “uninterrupted coding sequence”, i.e., lacking an intron, such as in a cDNA or it may include one or more introns bounded by appropriate splice junctions. An “intron” is a sequence of RNA which is contained in the primary transcript but which is removed through cleavage and re-ligation of the RNA within the cell to create the mature mRNA that can be translated into a protein.

“Constitutive promoter” refers to a promoter that is able to express the gene that it controls in all or nearly all of the plant tissues during all or nearly all developmental-stages of the plant, thereby generating “constitutive expression” of the gene.

“Co-suppression” and “sense suppression” refer to the production of sense RNA transcripts capable of suppressing the expression of identical or substantially identical transgene or endogenous genes.

“Contiguous” is used herein to mean nucleic acid sequences that are immediately preceding or following one another.

“Expression” refers to the transcription and stable accumulation of mRNA. Expression may also refer to the production of protein.

“Expression cassette” or “cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region may code for a protein of interest but may also code for a functional RNA of interest, for example antisense RNA or a nontranslated RNA, in the sense or antisense direction or both. The expression cassette comprising the nucleotide sequence of interest may be a chimeric sequence, meaning that at least one of its components is heterologous with respect to at least one of its other components.

As used herein, the terms “Fusarium graminearum”, “F. graminearum”, “Gibberella zeae”, and “G. zeae” have identical meaning and are used interchangeably to refer to the fungus species. The terms “Fusarium” and “Gibberella” have identical meaning and are used interchangeably to refer to the fungus genus.

“Gene” refers to a nucleic acid fragment that expresses mRNA, functional RNA, or specific protein, including regulatory sequences. The term “Native gene” refers to a gene as found in nature. The term “chimeric gene” refers to any gene that contains 1) DNA sequences, including regulatory and coding sequences, that are not found together in nature, or 2) sequences encoding parts of proteins not naturally adjoined, or 3) parts of promoters that are not naturally adjoined. Accordingly, a chimeric gene may comprise regulatory sequences and coding sequences that are derived from different sources, or comprise regulatory sequences and coding sequences derived from the same source, but arranged in a manner different from that found in nature. A “transgene” refers to a gene that has been introduced into the genome by transformation and is stably maintained. Transgenes may include, for example, genes that are either heterologous or homologous to the genes of a particular plant to be transformed. Additionally, transgenes may comprise native genes inserted into a non-native organism, or chimeric genes. The term “endogenous gene” refers to a native gene in its natural location in the genome of an organism. A “foreign” gene refers to a gene not normally found in the host organism but one that is introduced into the organism by gene transfer.

“Gene silencing” refers to homology-dependent suppression of pathogenicity genes, transgenes, or endogenous nuclear genes. Gene silencing may be transcriptional, when the suppression is due to decreased transcription of the affected genes, or post-transcriptional, when the suppression is due to increased turnover (degradation) of RNA species homologous to the affected genes.

“Genetically stable” and “heritable” refer to chromosomally-integrated genetic elements that are stably maintained in the plant and stably inherited by progeny through successive generations.

“Heterologous DNA Sequence” is a DNA sequence not naturally associated with a host cell into which it is introduced, including non-naturally occurring multiple copies of a naturally occurring DNA sequence.

The terms “micro RNA” and “miRNA” are used interchangeably herein. A miRNA is a stem-loop structure comprising a sense strand (called the “passenger strand”) and an antisense strand (called the “guide strand”). The miRNA is processed by a plant's endogenous DCL1-HYL1-SE protein complex, and it is the guide strand which hybridizes to the target RNA and drives the degradation mechanism.

The term “nucleic acid” refers to a polynucleotide of high molecular weight which can be single-stranded or double-stranded, composed of monomers (nucleotides) containing a sugar, phosphate and a base which is either a purine or pyrimidine. A “nucleic acid fragment” is a fraction of a given nucleic acid molecule. In higher plants, deoxyribonucleic acid (DNA) is the genetic material while ribonucleic acid (RNA) is involved in the transfer of information contained within DNA into proteins. A “genome” is the entire body of genetic material contained in each cell of an organism. The term “nucleotide sequence” refers to a polymer of DNA or RNA which can be single- or double-stranded, optionally containing synthetic, non-natural or altered nucleotide bases capable of incorporation into DNA or RNA polymers.

The terms “open reading frame” and “ORF” refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

“Operably-linked” and “Operatively-linked” refer to the association of nucleic acid sequences on a single nucleic acid fragment so that the function of one is affected by the other. For example, a promoter is operably-linked with a coding sequence or functional RNA when it is capable of affecting the expression of that coding sequence or functional RNA (i.e., that the coding sequence or functional RNA is under the transcriptional control of the promoter). Coding sequences in sense or antisense orientation can be operably-linked to regulatory sequences.

“Overexpression” refers to the level of expression in transgenic organisms that exceeds levels of expression in normal or untransformed organisms.

“Plant tissue” includes differentiated and undifferentiated tissues or plants, including but not limited to roots, stems, shoots, leaves, pollen, seeds, tumor tissue and various forms of cells and culture such as single cells, protoplast, embryos, and callus tissue. The plant tissue may be in plants or in organ, tissue or cell culture.

“Primary transformant” and “T₀ generation” refer to transgenic plants that are of the same genetic generation as the tissue that was initially transformed (i.e., not having gone through meiosis and fertilization since transformation). “Secondary transformants” and the “T₁, T₂, T₃, etc. generations” refer to transgenic plants derived from primary transformants through one or more meiotic and fertilization cycles. They may be derived by self-fertilization of primary or secondary transformants or crosses of primary or secondary transformants with other transformed or untransformed plants.

The terms “protein,” “peptide” and “polypeptide” are used interchangeably herein.

“Promoter” refers to a nucleotide sequence, which controls the expression of a coding sequence by providing the recognition for RNA polymerase and other factors required for proper transcription. “Promoter regulatory sequences” can comprise proximal and more distal upstream elements and/or downstream elements. Promoter regulatory sequences influence the transcription, RNA processing or stability, or translation of the associated coding sequence. Regulatory sequences include enhancers, untranslated leader sequences, introns, exons, and polyadenylation signal sequences. They include natural and synthetic sequences as well as sequences that can be a combination of synthetic and natural sequences. An “enhancer” is a nucleotide sequence that can stimulate promoter activity and can be an innate element of the promoter or a heterologous element inserted to enhance the level or tissue specificity of a promoter. The primary sequence can be present on either strand of a double-stranded DNA molecule, and is capable of functioning even when placed either upstream or downstream from the promoter. The meaning of the term “promoter” includes “promoter regulatory sequences.”

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived by posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA. A “functional RNA” refers to an antisense RNA, ribozyme, or other RNA that is not translated (but participates in a reaction or process as an RNA).

A “selectable marker gene” refers to a gene whose expression in a plant cell gives the cell a selective advantage. The selective advantage possessed by the cells transformed with the selectable marker gene may be due to their ability to grow in presence of a negative selective agent, such as an antibiotic or a herbicide, compared to the ability to grow of non-transformed cells. The selective advantage possessed by the transformed cells may also be due to their enhanced capacity, relative to non-transformed cells, to utilize an added compound as a nutrient, growth factor or energy source. A selective advantage possessed by a transformed cell may also be due to the loss of a previously possessed gene in what is called “negative selection”. In this, a compound is added that is toxic only to cells that did not lose a specific gene (a negative selectable marker gene) present in the parent cell (typically a transgene).

As used herein, “selfed” and “self-pollinated” are used interchangeably. Field crops are bred through techniques that take advantage of the plant's method of pollination. A plant is self-pollinated if pollen from one flower is transferred to the same or another flower of the same plant or a genetically identical plant. A plant is cross-pollinated if the pollen comes from a flower on a genetically different plant. Thus, the term “selfed” in a breeding program refers to self-pollination and the term “crossed” refers to cross-pollination.

The phrase “substantially identical,” in the context of two or more nucleic acid or protein sequences, refers to two or more sequences or subsequences that have at least 60%, preferably 80%, more preferably 90, even more preferably 95%, and most preferably at least 99% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In an especially preferred embodiment, the sequences are substantially identical over the entire length of the coding regions. Furthermore, substantially identical nucleic acid or protein sequences perform substantially the same function.

For sequence comparison, typically one sequence acts as a reference sequence to which test sequences are compared. When using a sequence comparison algorithm, “test” (or “query”) and “reference” (or “subject”) sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters. Those of skill in the art understand that to avoid a high similarity to a reference sequence due to inclusion of gaps in the polynucleotide sequence a gap penalty is typically introduced and is subtracted from the number of matches.

Optimal alignment of sequences for comparison can be conducted, e.g., by the local homology algorithm of Smith & Waterman, 1981, Adv. Appl. Math. 2: 482, by the homology alignment algorithm of Needleman & Wunsch, 1970, J. Mol. Biol. 48: 443, by the search for similarity method of Pearson & Lipman, 1988, Proc. Nat'l. Acad. Sci. 85: 2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel et al., infra).

One example of an algorithm that is suitable for determining percent sequence identity and sequence similarity is the BLAST algorithm, which is described in Altschul et al., 1990, J. Mol. Biol. 215: 403-410. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, 1989, Proc. Natl. Acad. Sci. 89: 10915).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

For purposes of the present invention, comparison of nucleotide sequences for determination of percent sequence identity to the promoter sequences disclosed herein is preferably made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

Another indication that two nucleic acid sequences are substantially identical is that the two molecules hybridize to each other under stringent hybridization conditions. The phrase “hybridizing specifically to” refers to the binding, duplexing, or hybridizing of a molecule only to a particular nucleotide sequence under stringent hybridization conditions when that sequence is present in a complex mixture (e.g., total cellular) of DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part I chapter 2, “Overview of principles of hybridization and the strategy of nucleic acid probe assays”, Elsevier, New York. Generally, high stringency hybridization and wash conditions are selected to be about 5° C. lower than the thermal melting point (Tₘ) for the specific sequence at a defined ionic strength and pH. Typically, under high stringency conditions a probe will hybridize to its target subsequence, but to no other sequences.

The Tₘ is the temperature (under defined ionic strength and pH) at which 50% of the target sequence hybridizes to a perfectly matched probe. Very high stringency conditions are selected to be equal to the Tₘ for a particular probe. An example of high stringency hybridization conditions for hybridization of complementary nucleic acids which have more than 100 complementary residues on a filter in a Southern or northern blot is 50% formamide with 1 mg of heparin at 42° C., with the hybridization being carried out overnight. An example of very high stringency wash conditions is 0.1 5M NaCl at 72° C. for about 15 minutes. An example of high stringency wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), high stringency conditions typically involve salt concentrations of less than about 1.0 M Na ion, typically about 0.01 to 1.0 M Na ion concentration (or other salts) at pH 7.0 to 8.3, and the temperature is typically at least about 30° C. High stringency conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under high stringency conditions are still substantially identical if the proteins that they encode are substantially identical. This occurs, for example, when a copy of a nucleic acid is created using the maximum codon degeneracy permitted by the genetic code.

Low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium. citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

The following are examples of sets of hybridization/wash conditions that may be used to clone homologous nucleotide sequences that are substantially identical to reference nucleotide sequences of the present invention: a reference nucleotide sequence hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., or alternately in 7% sodium dodecyl sulfate (SDS), 0.5 M NaPO₄, 1 mM EDTA at 50° C. with washing in 1×SSC, 0.1% SDS at 50° C., or alternately still in 7% sodium dodecyl sulfate (SDS), 0.5 M NaPO₄, 1 mM EDTA at 50° C. with washing in 0.5×SSC, 0.1% SDS at 50° C., or alternately in 7% sodium dodecyl sulfate (SDS), 0.5 M NaPO₄, 1 mM EDTA at 50° C. with washing in 0.1×SSC, 0.1% SDS at 50° C., or alternately in 7% sodium dodecyl sulfate (SDS), 0.5 M NaPO₄, 1 mM EDTA at 50° C. with washing in 0.1×SSC, 0.1% SDS at 65° C.

Specificity is typically the function of post-hybridization washes, the critical factors being the ionic strength and temperature of the final wash solution. For example, if sequences with >90% identity are sought, the Tₘ can be decreased 10° C. Generally, high stringency conditions are selected to be about 19° C. lower than the Tₘ for the specific sequence and its complement at a defined ionic strength and pH. However, very high stringency conditions can utilize a hybridization and/or wash at 1, 2, 3, or 4° C. lower than the Tₘ; moderately stringent conditions can utilize a hybridization and/or wash at 6, 7, 8, 9, or 10° C. lower than the Tₘ; low stringency conditions can utilize a hybridization and/or wash at 11, 12, 13, 14, 15, or 20° C. lower than the T. Using the equation, hybridization and wash compositions, and desired temperature, those of ordinary skill will understand that variations in the stringency of hybridization and/or wash solutions are inherently described. If the desired degree of mismatching results in a temperature of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology-Hybridization with Nucleic Acid Probes, Part 1, Chapter 2 (Elsevier, New York); and Ausubel et al., eds. (1995) Current Protocols in Molecular Biology, Chapter 2 (Greene Publishing and Wiley—Interscience, New York). See Sambrook et al. (1989) Molecular Cloning: A Laboratory Manual (2d ed., Cold Spring Harbor Laboratory Press, Plainview, N.Y.).

The term “transformation” refers to the transfer of a nucleic acid fragment into the genome of a host cell, resulting in genetically stable inheritance. “Transiently transformed” refers to cells in which transgenes and foreign DNA have been introduced (for example, by such methods as Agrobacterium-mediated transformation or biolistic bombardment), but not selected for stable maintenance. “Stably transformed” refers to cells that have been selected and regenerated on a selection media following transformation.

“Transformed/transgenic/recombinant” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed”, “non-transgenic”, or “non-recombinant” host refers to a wild-type organism, e.g., a bacterium or plant, which does not contain the heterologous nucleic acid molecule.

“Transient expression” refers to expression in cells in which a virus or a transgene is introduced by viral infection or by such methods as Agrobacterium-mediated transformation, electroporation, or biolistic bombardment, but not selected for its stable maintenance.

“Vector” is defined to include, inter alia, any plasmid, cosmid, phage or Agrobacterium binary vector in double or single stranded linear or circular form which may or may not be self transmissible or mobilizable, and which can transform prokaryotic or eukaryotic host either by integration into the cellular genome or exist extrachromosomally (e.g. autonomous replicating plasmid with an origin of replication). Specifically included are shuttle vectors by which is meant a DNA vehicle capable, naturally or by design, of replication in two different host organisms, which may be selected from actinomycetes and related species, bacteria and eucaryotic (e.g. higher plant, mammalian, yeast or fungal cells).

“Visible marker” refers to a gene whose expression does not confer an advantage to a transformed cell but can be made detectable or visible. Examples of visible markers include but are not limited to β-glucuronidase (GUS), luciferase (LUC) and green fluorescent protein (GFP).

“Wild-type” refers to the normal gene, virus, or organism found in nature without any known mutation.

### DRAWINGS

FIG. 1 illustrates the expression cassette comprising the dual tandem array FgRNA miRNA stem-loop coding regions (2 & 3), their passenger and guide sequences (5 & 6, and 7 & 8, respectively), and their positions relative to each other, to the promoter (1), and to the terminator (4). In one aspect of the present invention, 5 may be represented by SEQ ID NO: 3, and 6 may be represented by SEQ ID NO: 4; 7 may be represented by SEQ ID NO: 1, and 8 may be represented by SEQ ID NO: 2.

FIG. 2 illustrates the stem-loop formed when the micro RNA folds back on itself, prior to processing by the cell. FIG. 2a depicts the endogenous soybean miR319 (SEQ ID NO: 12). FIG. 2b depicts the FgRNA-2 (SEQ ID NO: 17) comprising SEQ ID NOs: 3 and 4. FIG. 2c depicts the FgRNA-1 (SEQ ID NO: 16) comprising SEQ ID NOs: 1 and 2. Stem-loop folding calculated according to Michael Zuker (Mfold web server for nucleic acid folding and hybridization prediction. Nucleic Acids Res. 31 (13), 3406-3415, 2003; http://mfold.rna.albany.edu/?q=mfold/mfold-references).

### DETAILED DESCRIPTION

This invention relates to nucleic acid sequences, preferably isolated nucleic acid sequences, which confer resistance to fungal disease upon host plants. This invention is also drawn to plants expressing the nucleic acid sequences, whereby the plants are resistant to fungal disease. These plants which express these nucleic acid sequences are useful in controlling fungal disease caused by a pathogenic fungus, particularly a Fusarium species, and more particularly Fusarium graminearum.

In one embodiment, the present invention comprises a single-stranded nucleic acid molecule, or an isolated single-stranded nucleic acid molecule, comprising a first sequence and a second sequence, wherein the first sequence comprises a sequence obtained from a gene that encodes a fungal ribosomal RNA, and the second sequence comprises a sequence capable of forming a duplex with the first sequence. In another embodiment, the fungal ribosomal RNA is from a fungus in the genus Fusarium. In another embodiment, the fungal ribosomal RNA is the 28S ribosome from Fusarium graminearum. In another embodiment, the first sequence is selected from the group consisting of SEQ ID NOs: 1, 3, 5, and 7. In another embodiment, the second sequence is selected from the group consisting of SEQ ID NOs: 2, 4, 6, and 8. In another embodiment, the single-stranded nucleic acid molecule further comprises a backbone sequence between the first sequence and the second sequence. In another embodiment, the backbone sequence comprises at least nucleotides 41 to 167 of SEQ ID NO: 12. In another embodiment, the single-stranded nucleic acid sequence is capable of forming a hairpin. In another embodiment, the single-stranded nucleic acid molecule is synthetic. In another embodiment, the nucleic acid is RNA or DNA or an DNA/RNA hybrid. In yet another embodiment, the single-stranded nuckeic acid molecule of the invention is active against a Fusarium fungus or a Phakopsora fungus. In another embodiment, the Fusarium fungus is Fusarium graminearum. In another embodiment, the Phakopsora fungus is Phakopsora pachyrhizi.

In another embodiment, the present invention comprises an expression cassette comprising at least a first nucleic acid sequence which encodes for a first single-stranded nucleic acid molecule comprising a first sequence and a second sequence, wherein the first sequence comprises a sequence obtained from a gene that encodes a fungal ribosomal RNA, and the second sequence comprises a sequence capable of forming a duplex with the first sequence. In another embodiment, the expression cassette further comprises a second nucleic acid sequence, wherein the first single-stranded molecule and the second single-stranded molecule do no comprise identical first sequences. In another embodiment, the first single-stranded molecule comprises a first sequence selected from the group consisting of SEQ ID NOs: 1, 3, 5, and 7, and the second single-stranded molecule comprises a first sequence different from the first sequence in the first single-stranded molecule. In another embodiment, the first single-stranded molecule comprises a first sequence comprising SEQ ID NO: 1, and the second single-stranded molecule comprises a first sequence comprising SEQ ID NO: 3. In another embodiment, the expression cassette comprises SEQ ID NO: 13.

In another embodiment, the present invention comprises a vector comprising an expression cassette comprising at least a first nucleic acid sequence which encodes for a first single-stranded nucleic acid molecule comprising a first sequence and a second sequence, wherein the first sequence comprises a sequence obtained from a gene that encodes a fungal ribosomal RNA, and the second sequence comprises a sequence capable of forming a duplex with the first sequence. In another embodiment, the vector comprises SEQ ID NO: 14 or 15.

In another embodiment, the present invention comprises a non-human host cell comprising an expression cassette comprising at least a first nucleic acid sequence which encodes for a first single-stranded nucleic acid molecule comprising a first sequence and a second sequence, wherein the first sequence comprises a sequence obtained from a fungal ribosome, and the second sequence comprises a sequence capable of forming a duplex with the first sequence. In another embodiment, the non-human host cell is selected from the group consisting of bacteria, virus, fungus, plant, and animal cells. In another embodiment, the non-human host cell is a plant cell.

In another embodiment, the present invention comprises a plant comprising a plant cell comprising an expression cassette comprising at least a first nucleic acid sequence which encodes for a first single-stranded nucleic acid molecule comprising a first sequence and a second sequence, wherein the first sequence comprises a sequence obtained from a gene that encodes a fungal ribosomal RNA and the second sequence comprises a sequence capable of forming a duplex with the first sequence. In another embodiment, the plant is a monocot. In another embodiment, the monocot is maize. In another embodiment, the plant is a dicot. In another embodiment, the dicot is soybean. In yet another embodiment, the transgenic plant of the invention is resistant to a Fusarium fungus or a Phakopsora fungus. In another embodiment, the Fusarium fungus is Fusarium graminearum. In another embodiment, the Phakopsora fungus is Phakopsora pachyrhizi.

In another embodiment, the present invention comprises a method of producing a plant resistant to a fungal pathogen, comprising the steps of: (a) obtaining an expression cassette comprising a nucleotide sequence encoding a single-stranded nucleic acid molecule, or an isolated single-stranded nucleic acid molecule, comprising a first sequence and a second sequence, wherein the first sequence comprises a sequence obtained from a gene that encodes a fungal ribosomal RNA, and the second sequence comprises a sequence capable of forming a duplex with the first sequence; (b) inserting the expression cassette into the genome of a plant cell; and (c) generating a plant from the plant cell; wherein the plant is resistant to a fungal pathogen. In another embodiment, the isolated single-stranded nucleic acid molecule comprises a first sequence selected from the group consisting of SEQ ID NOs: 1, 3, 5, and 7. In another embodiment, the plant cell is a maize plant cell. In another embodiment, the plant is a maize plant. In another embodiment, the plant cell is a soybean plant cell. In another embodiment, the plant is a soybean plant. In another embodiment, a method of the inventionprodecues a plant that is resistant to a Fusarium fungus or a Phakopsora fungus. In another embodiment, the Fusarium fungus is Fusarium graminearum. In another embodiment, the Phakopsora fungus is Phakopsora pachyrhizi.

All the features disclosed in this specification (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

These embodiments are better understood in light of the Examples provided below.

### Example 1

### Discovering & Cloning miRNA Targets

Potential micro RNA (miRNA) targets were identified from scans of genomic DNA encoding the 28S ribosomal RNA (rRNA) derived from the maize fungal pathogen Fusarium graminearum (also known as Gibberella zeae).

The genomic DNA sequence encoding 28S ribosomal RNA from Fusarium graminearum harbors a 125 bp sequence that when expressed as an RNA duplex exhibits high in vitro anti-fungal activity as measured by inhibition of spore germination. SEQ ID NO: 11 shows a partial sequence of the Fusarium graminearum 28S ribosomal RNA gene (LOCUS: AY188924). The sequence from nucleotides 476-600 has in vitro anti-fungal activity as an RNA duplex). Four passenger miRNA sequences (SEQ ID NOs: 18, 19, 5, and 7), and their antisense guide sequences (SEQ ID NOs: 2, 4, 6, and 8) were identified (Table 1) based on the partial sequence encoding the 28S ribosomal RNA and selected for further testing.

TABLE 1
Passenger and Guide Strands of FgRNA miRNA
molecules

| | | Sequence (5′ to 3′ |
|---|---|---|
| SEQ Number | Name | direction) |
| SEQ ID NO: 18 | FgRNA-1 | nnCCUCGGAUCAGGUAGGAAU |
| | passenger | |
| SEQ ID NO: 2 | FgRNA-1 | AUUCCUACCUGAUCCGAGGnn |
| | guide | |
| SEQ ID NO: 19 | FgRNA-2 | nnCCGCUGAACUUAAGCAUAU |
| | passenger | |
| SEQ ID NO: 4 | FgRNA-2 | AUAUGCUUAAGUUCAGCGGnn |
| | guide | |
| SEQ ID NO: 5 | FgRNA-3 | nnAUAUCAAUAAGCGGAGGAA |
| | passenger | |
| SEQ ID NO: 6 | FgRNA-3 | UUCCUCCGCUUAUUGAUAUnn |
| | guide | |
| SEQ ID NO: 7 | FgRNA-4 | nnCCUAGUAACGGCGAGUGAA |
| | passenger | |
| SEQ ID NO: 8 | FgRNA-4 | UUCACUCGCCGUUACUAGGnn |
| | guide | |

In each of the sequences in Table 1, a double nucleotide overhang, herein represented as “nn”, is included on the 3′ end. The overhang is needed for Dicer to process the duplex. As used herein, “n” is meant to represent any nucleotide. Therefore, any nucleotide, or any combination of nucleotides, can be used in the overhang. In one aspect, any combination of A, T, U, G, or C is used in the overhang. In another aspect, the same nucleotide is used twice in the overhang. In another aspect, the overhang is TT or UU.

The antisense guide strands are responsible for driving the RNA degradation mechanism within the plant.

### Example 2

### In Vitro Bioassays

Novel synthetic RNA duplexes comprising at least two passenger sequences selected from the group consisting of SEQ ID NOs: 1-4 were created and tested by in vitro bioassays against F. graminearum and the soybean rust pathogen (Phakopsora pachyrhizi).

Synthetic RNA duplexes were created and tested by in vitro bioassays. These assays are described in U.S. Patent Application Publication No. 2010/0257634 A1 (Ser. No. 12/753,901), incorporated herein by reference in its entirety. Approximately 10 μg of these individual RNA duplexes were incubated with spores of the soybean rust fungus (Phakopsora pachyrhizi) then assessed for anti-fungal activity as measured by percent inhibition of germination and appressorium formation. Data shown in Table 2 indicate that RNA duplex FgRNA-1 (comprising SEQ ID NOs: 18 & 2) and RNA duplex FgRNA-2 (comprising SEQ ID NOs: 19 & 4) rate the highest level of inhibition. RNA duplex FgRNA-3 (comprising SEQ ID NOs: 5 & 6) and RNA duplex FgRNA-4 (comprising SEQ ID NOs: 7 & 8) have a moderate level of inhibitory activity. Importantly, the negative control (FgRNA-5), which comprises nonsense RNA sequences SEQ ID NOs: 9 & 10, had virtually no affect on spore germination or appressorium formation.

TABLE 2
Percent Inhibition Results from in vitro Bioassay Tests of RNA duplexes
against P. pachyrhizi.

% Inhibition

| RNA duplex: | Trial 1 | Trial 2 |
|---|---|---|
| FgRNA-1 | 94 | 93 |
| FgRNA-2 | 89 | 81 |
| FgRNA-3 | 48 | 41 |
| FgRNA-4 | 50 | 42 |
| FgRNA-5 | 5 | 8 |

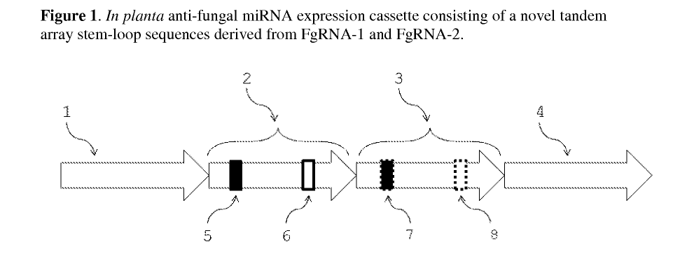

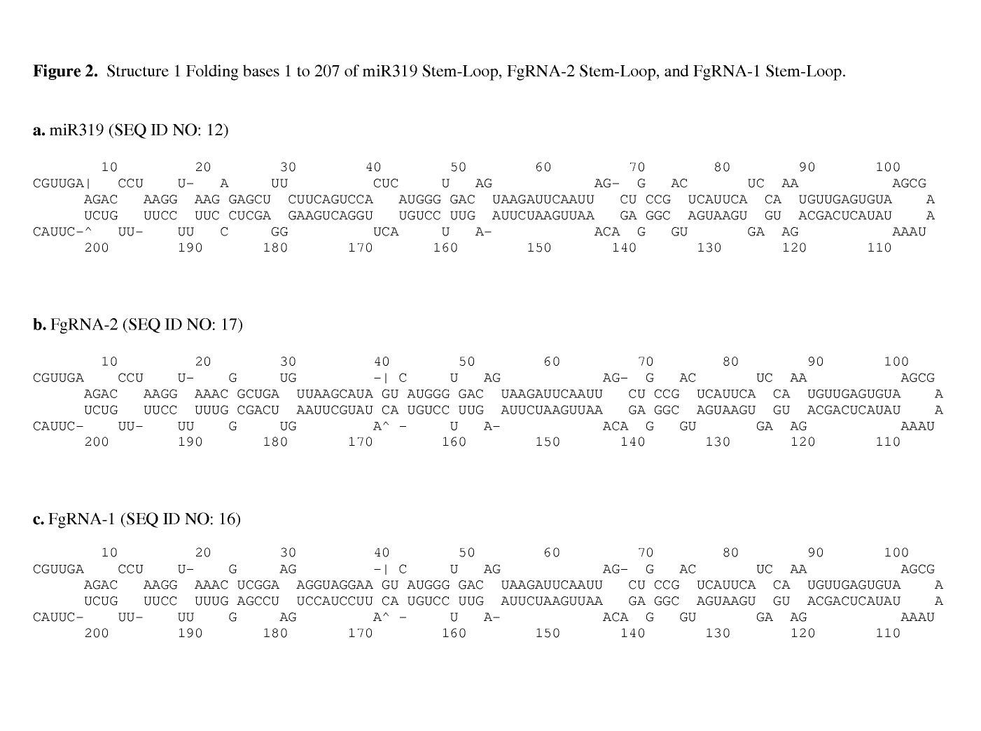

Based on these data, synthetic miRNA in planta expression cassettes were created based on the endogenous soybean micro-RNA miR319, SEQ ID NO: 12 (Subramanian, et al. 2008). Being the better performing duplexes, FgRNA-1 and FgRNA-2 were chosen for further development. The passenger strands for FgRNA-1 and FgRNA-2 were engineered so that each would mimic the folding and mismatches that miR319 possesses when folded. Therefore, SEQ ID NO: 18 was engineered to become SEQ ID NO: 1, and SEQ ID NO: 19 was engineered to become SEQ ID NO: 3. The passenger and guide strand sequences of miR319 (nucleotides 21-40 for passenger and 169-188 for guide of SEQ ID NO: 12) were replaced by those sequences derived from FgRNA-1 (comprising SEQ ID NOs: 1 & 2). The passenger and guide strand sequences of miR319 (nucleotides 21-40 for passenger and 169-188 for guide of SEQ ID NO: 12) were replaced by those sequences derived from FgRNA-2 (comprising SEQ ID NOs: 3 & 4). See FIG. 2, which shows the folded stem-loop of miR319 (FIG. 2a), FgRNA-2 (FIG. 2b), and FgRNA-1 (FIG. 2c). These miR319 derived expression elements were linked in a novel tandem dual-expression array to a Cestrum viral promoter and a NOS terminator (FIG. 1). Subsequently, the plant expression cassette was ligated to binary vectors for soybean or maize transformation (SEQ ID NOs: 14 and 15, respectively). These synthetic micro RNAs stem-loop structures are then processed by the plants endogenous DCL1-HYL1-SE protein complex (Dong, et al. 2008. PNAS 105(29):9970-9975) to produce the anti-fungal miRNAs.

These bioassays confirmed the ability of these RNA duplexes to prevent germination of fungal spores.

### Example 3

### Maize & Soybean Transformation

To express these anti-fungal duplexes in planta, novel miRNA gene expression cassettes were created for transformation of maize and soybean. The passenger and guide sequences of the soybean endogenous miR319 stem-loop were modified, stacked in a duplex and used to create maize and soybean transformation vectors, as shown above.

Maize and soybean transformation experiments were initiated with mannose or hygromycin selection, respectively. The Tₒ-generation events were analyzed by qRT-PCR assays for the presence of guide strand miRNAs derived from FgRNA-1 and FgRNA-2. The qRT-PCT assay is sensitive and accurate for determining transcript levels of RNA. Briefly, RNA is purified from tissue samples and the target sequence is reverse transcribed into a DNA molecule. A reference RNA molecule (usually of a constitutively expressed gene, such as elongation factor Efla) is also reverse transcribed for control purposes. The DNA molecules for the target sequence and the reference sequence are then amplified using Real-Time PCR. Relative expression levels are determined by comparing the cycle threshold (Ct) of the target sequence and the reference sequence. Results from the qRT-PCR analysis proved that both miRNAs are in fact expressed in both T₀-generation soybean and corn events (Table 3).

TABLE 3
Relative expression levels as measured by qRT-PCR analysis of T₀-
generation events expressing synthetic anti-fungal mi-RNAs targeting
F. graminearum ribosomal RNAs.

| Plant ID | Crop | FgRNA-1 | Std. Error | FgRNA-2 | Std. Error |
|---|---|---|---|---|---|
| SYA002A | soybean | 36.61 | 3.95 | 154.82 | 5.58 |
| MZA006A | maize | 128.24 | 7.22 | 1295.31 | 147.98 |
| MZA010A | maize | 90.65 | 14.11 | 1564.17 | 209.73 |
| MZA021A | maize | 25.86 | 4 | 1126.68 | 272.67 |
| MZA035A | maize | 1024.67 | 176.48 | 4248.15 | 728.28 |
| MZA037A | maize | 33.67 | 3.5 | 1107.27 | 145.64 |
| MZA038A | maize | 3529.38 | 487.32 | 7828.97 | 1385.62 |
| MZA042A | maize | 21.83 | 6.38 | 1454.4 | 76.57 |
| MZB008A | maize | 16.98 | 3.69 | 793.27 | 76.62 |
| MZB011A | maize | 24.61 | 3.87 | 1239.19 | 136.6 |
| MZB012A | maize | 95.2 | 8.47 | 3558.43 | 591.5 |
| MZB018A | maize | 17.69 | 1.99 | 301.96 | 40.16 |
| MZB024A | maize | 63.39 | 8.47 | 3060.78 | 276.86 |
| MZB025A | maize | 62.34 | 6.38 | 1886.2 | 302.71 |
| MZB028A | maize | 83.73 | 9.11 | 845.39 | 68.29 |
| MZB031A | maize | 131.05 | 10.14 | 2783.75 | 477.45 |
| MZB033A | maize | 86.31 | 6.71 | no data | no data |
| MZB034A | maize | 10.14 | 1.98 | 422.05 | 67.4 |
| MZB037A | maize | 150.54 | 20.15 | 111.61 | 7.63 |
| MZB038A | maize | 55.93 | 10.58 | 1026.72 | 192.94 |
| MZC001A | maize | 984.62 | 69.27 | 2961.87 | 397.87 |
| MZC010A | maize | 78.79 | 13.43 | 2846.36 | 639.4 |
| MZC015A | maize | 329.49 | 11.26 | 5025.25 | 606.97 |

The analysis of T₀-generation maize and soybean events by qRT-PCR confirmed the expression of specific anti-fungal guide strand sequences. This is the first demonstration of cross-species expression of synthetic miRNA in maize and soybean. Further, this proves that miRNA can be expressed in planta by a single expression cassette as a duplex and that a dual-tandem array in a single expression cassette can be processed correctly. Interestingly, the stem-loop comprising FgRNA-2, which is closer to the promoter, is detected at a much higher level than FgRNA-1.

These events listed in Table 3 were self-pollinated to create the T₁-generation of seed for further testing. Unfortunately, the single soybean event was a chimeric plant, and therefore, the trait was not inherited in the next generation. However, ten of the maize events were successfully selfed. The T₁-generation plants were sampled for zygosity analysis followed by qRT-PCR.

TABLE 4
The results from qRT-PCR analysis of the T₁-generation maize events
expressing anti-fungal miRNAs.

| Plant ID | FgRNA-1 | Std. Error | FgRNA-2 | Std. Error |
|---|---|---|---|---|
| MWB00400622 | 1389.99 | 260.46 | 12430.14 | 2459.25 |
| MWB00400626 | 473.78 | 54.95 | 6556.82 | 446.81 |
| MWB00400661 | 336.62 | 69.04 | 4609.13 | 639.45 |
| MWB00400680 | 588.4 | 74.76 | 8060.3 | 1248.23 |
| MWB00400692 | 1293.76 | 155.88 | 13904.11 | 1999.42 |
| MWB00400697 | 564.05 | 18.01 | 9289.91 | 831.72 |
| MWB00400567 | 739.37 | 99.71 | 8192.48 | 1141.92 |
| MWB00400584 | 319.21 | 136.28 | 7629.19 | 1931.2 |
| MWB00400594 | 703.28 | 80.69 | 8565.13 | 543.64 |
| MWB00400600 | 282.27 | 97.98 | 5324.47 | 735.05 |
| MWB00400646 | 355.2 | 44.42 | 5089.37 | 400.97 |
| MWB00400654 | 910.71 | 127.18 | 9169.79 | 1691.43 |

The analysis of the T₁-generation plants, summarized in Table 4, found those plants, whether homozygous or heterozygous, derived from independent events expressed the anti-fungal miRNAs. As expected, the null-siblings were negative for FgRNA-1 and FgRNA-2 expression as determined by qRT-PCR. Consistent with the T₀ generation analysis, the FgRNA-2 which is closer to the promoter is expressed at a much higher level than FgRNA-1 in the T₁-generation analysis.

### Example 4

### Disease Severity Testing

Greenhouse experiments confirmed expression of these miRNA in T₁-generation maize lines. Molecular characterization identified homozygous, heterozygous and null-sibling maize plants. These plants were used in detached leaf bioassays by inoculation with spores of F. graminearum. At 10 days post-inoculation, leaves were rated for disease severity. Results show improved tolerance to F. graminearum on leaves taken from homozygous or heterozygous plants compared to either null-sibs or non-transgenic maize leaves.

The FgRNA-1 and FgRNA-2 guide strands were originally identified by in vitro bioassays for their ability to prevent F. graminearum spore germination at a rate of >90% efficacy (Table 5.). Subsequently, detached leaf bioassays were performed on T₁-generation (homozygous or heterozygous) that tested positive by qRT-PCR. Leaves were collected, placed in a humidity chamber followed by inoculation with F. graminearum spores (25,000 spores/ml). Leaves from null-siblings or non-transgenic maize served as negative controls.

TABLE 5
Percent Inhibition by RNA duplexes at three different concentrations

| | Percent Inhibition | | |
|---|---|---|---|
| | (at Given Concentration | | |
| | of RNA duplex) | | |
| RNA duplex | 50 μg | 35 μg | 25 μg |
| FgRNA-1 | 97 | 94 | 66 |
| FgRNA-2 | 75 | 79 | 61 |
| FgRNA-3 | 84 | 96 | 54 |
| FgRNA-4 | 93 | 90 | 58 |
| FgRNA-5 | 68 | 68 | 17 |
| Mixture of all 5 | 100 | 100 | 100 |

A disease severity rating was used to rate each of the individual leaves tested in these experiments 6-10 days post inoculation (0=no disease, 1=trace, 2=low, 3=intermediate, and 4=severe). See Table 6. The null-siblings or nontransgenic leaves showed intermediate or severe levels of disease, while most leaves expressing FgRNA-1 and FgRNA-2 showed no signs of disease.

TABLE 6
Detached Leaf Assays.

| | | | | Disease Ratings of Detached | | |
|---|---|---|---|---|---|---|
| | Zygosity | | qRT- | Leaf Assays | | |
| Maize Plant ID | Promoter | Marker | PCR | G. zeae | C. graminicola | G. moniliformis |
| MWB00400619 | Hom | Hom | + | 0 | 3 | 0 |
| MWB00400614 | Hom | Hom | + | 2 | 4 | 4 |
| MWB00400658 | Het | Het | + | 0 | 2 | 2 |
| MWB00400574 | Hom | Hom | + | 0 | 2 | 2 |
| MWB00400604 | Het | Het | + | 0 | 3 | 3 |
| MWB00400582 | Het | Het | + | 0 | 4 | 4 |
| MWB00400581 | 0 | 0 | − | 0 | 4 | 4 |
| MWB00400576 | 0 | 0 | − | 2 | 3 | 4 |
| MWB00400686 | 0 | 0 | − | 2 | 4 | 2 |
| MWB00400588 | 0 | 0 | − | 3 | 4 | 3 |
| MWB00400664 | Hom | Hom | + | 4 | 4 | N/A |

In a second experiment, leaves from these same events were inoculated with the maize anthracnose pathogen Colletotrichum graminicola (anamorph Glomerella graminicola) at 25,000 spores/ml. None of these events expressing FgRNA-1 and FgRNA-2 have tolerance to this fungal pathogen when rated 6-10 days post inoculation. This experiment demonstrates the specificity of the anti-fungal miRNAs targeting F. graminearum. Secondly, the positive results observed are most likely not the result of the activation of endogenous maize disease resistance mechanisms.

### Example 5

### T₂ Plants

The T₁-generation homozygous and null-siblings from independent maize events comprising SEQ ID NO: 13 were selfed to increase T₂ seed. Ragdoll bioassays were performed on the T₂ seed. A ragdoll bioassay test consisted of 10 seeds spaced in a line 10 cm from the top edge of a layer of 31 cm by 61 cm germination paper and moistened with distilled, deionized H₂O. A spore suspension of consisting of macroconidia of Fusarium graminearum, quantified to 1×10⁶ spores/ml, is dropped onto each seed with a dropper. A second pre-moistened sheet of germination paper was placed over the first layer, and the entire assembly was rolled along the short axis and secured with rubber bands. Units were placed in a plastic bag and incubated vertically in an incubator for approximately 72 hours at 12° C. (assay parameters were adjusted to give maximum root discoloration without killing the plants, therefore, time in the incubator varied; too much disease meant less time in the 12° C. incubator), then moved to another incubator for 96 hours at 25° C. in the light (for 16 hour intervals) and 20° C. in the dark (8 hour intervals). At the end of the incubation period, bioassay units were unrolled, and root discoloration and germination rates were recorded in Table 7, below.

TABLE 7
Ragdoll Bioassays on T₂ inbred maize seeds.

| | | Total Seeds | % | | |
|---|---|---|---|---|---|
| Plant ID | Ragdoll No. | Planted | Germination | % Healthy | Other Observations |
| 11SBI001996 | 1 | 10 | 20 | 0 | |
| 11SBI002000 | 2 | 10 | 50 | 0 | |
| 11SBI002021 | 3 | 10 | 90 | 0 | Penicillium contamination |
| 11SBI002036 | 4 | 10 | 90 | 0 | Penicillium contamination |
| 11SBI002038 | 5 | 10 | 30 | 0 | |
| 11SBI002041 | 6 | 10 | 10 | 0 | |
| 11SBI002042 | 7 | 10 | 30 | 0 | |
| 11SBI002043 | 8 | 10 | 40 | 0 | |
| 11SBI003020 | 9 | 10 | 10 | 0 | |
| 11SBI003023 | 10 | 10 | 20 | 0 | |
| 11SBI003024 | 11 | 10 | | | Tricoderma contamination - ragdoll discarded |
| 11SBI003029 | 12 | 10 | 80 | 12.5 | |
| 11SBI003033 | 13 | 10 | 80 | 0 | |
| 11SBI003038 | 14 | 10 | 90 | 0 | |
| 09MZ000080 | 15 | 10 | 80 | 0 | Postive Control: Inoculated with F. graminearum |
| 09MZ000084 | 16 | 10 | 100 | 40 | Postive Control: Inoculated with F. graminearum |
| 09MZ000080 | 17 | 10 | 90 | 100 | Negative Control: not inoculated |
| 09MZ000084 | 18 | 10 | 100 | 100 | Negative Control: not inoculated |

Control maize seeds from hybrid lines 09MZ000080 and 09MZ000084 were included in ragdolls 15-18. The control seeds performed better due to the fact that they represent a hybrid genetic background, whereas the test T₂ seeds (in ragdolls 1-14) represent inbred lines which were expected to perform poorly. It is submitted that the poor results are due to the lines being inbred and not to the performance of FgRNA-1 and FgRNA-2 stem-loops. This is supported by the generally poor germination rate, as observed during the ragdoll bioassays. Additionally problematic is the contamination by Penicillium in ragdolls 3 and 4, and Tricoderma in ragdoll 11.

T₂ seeds comprising SEQ ID NO: 13 are backcrossed into a hybrid maize genetic background. These seeds display an increased resistance to disease caused by Fusarium graminearum due to the expression of FgRNA-1 and FgRNA-2 stem-loops.

### Example 6

### Soybean Plants Resistant to Soybean Rust

A second round of soybean transformation experimnets were carried out using the FgRNA-1 and FgRNA-2 miRNA molecules described above. Tₒ-generation events were analyzed for the presence of guide strand miRNAs derived from FgRNA-1 and FgRNA-2 as described above. Relative expression levels of the specific guide strands were comparable to the levels disclosed in Table 3. Positive T₀ events were self pollinated to create the T₁-generation of seed. Plants grown from T₁ seed were sampled for zygosity analysis followed qRT-PCR as cdescribed above. Eight of the highest expressing events were selected for generation of T₂ seed and for testing against soybean fungal diseases.

The transgenic T2 soybean plants expressing the FgRNA-1 and FgRNA-2 miRNA molecules were evaluated for resistance to the fungus Phakopsora pachyrhizi, the causative agent of soybean rust disease. Soybean rust spores were collected from inoculated leaves of non-transgenic susceptible soybean variety “JACK” by washing leaves in water plus 0.01% Tween 20. The spore concentration was adjusted to about 500,000 spores per ml. Plants from the transgenic soybean lines expressing FgRNA-1 and FgRNA-2 were inoculated at the V-2 stage. At about 10-14 days post inoculation, the first trifoliate leaf was rated (scale 0-100%) for disease severity.

Three separate whole-plant trials were carried out in a greenhouse. In the first experiment, some of the events showed a decrease in disease severity. However, the incidence of disease was low in all treatments including the susceptible control soybean line (JACK), i.e. there was not adequate spore germination to have a conclusive test. Data from two further trials showed that some plants in all the transgenic events had reduced disease compared to the non-transgenic control (soybean variety Jack). In addition, in the second trial all plants from two events (4B001A104 and 11B004A218) had reduced disease and in the third trial all plants from three events (4B001A104, 11B004A218 and 6B001A149) had reduced levels of disease compared to the non-transgenic control (soybean variety Jack). The results of trials 2 and 3 are shown in Table 8. The “% disease” column indicates the percent of leaf area infected. The “% control” column indicates the reduction in disease compared to the susceptible control. These results clearly show FgRNA-1 and FgRNA-2, which were designed to target 28s ribosomal RNA in Fusarium graminearum, also target the 28s ribosomal RNA in Phakopsora pachyrhizi. Thus, such RNAi molecules have utility in multiple crops to target and control multiple diseases. Such RNAi molecules are particularly useful in controlling a Fusarium fungus, for example Fusarium graminearum, or a Phakopsora fungus, for example Phakopsora pachyrhizi.

TABLE 8
Results of whole plant greenhouse tests of resistance transgenic soybean events to
Phakopsora pachyrhizi.

| | Test 2 | | | | Test 3 | | | |
|---|---|---|---|---|---|---|---|---|
| Event | # plants | % disease | Range | % control | # plants | % disease | Range | % control |
| 4B001A104 | 10 | 39.0 | 22-63 | 47.1 | 18 | 42.3 | 27-52 | 38.2 |
| 11B004A218 | 10 | 38.1 | 14-62 | 48.3 | 18 | 34.0 | 20-42 | 50.3 |
| 6B001A149 | 10 | 61.5 | 40-85 | 25.6 | 17 | 40.0 | 28-57 | 41.5 |
| 6B004A157- | 11 | 70.9 | 50-82 | 3.8 | | | | |
| 1A004A218 | 12 | 75.7 | 70-80 | 0.0 | | | | |
| 18A001A221 | 11 | 74.7 | 48-87 | 0.0 | | | | |
| 18A003A238 | 6 | 75.8 | 72-80 | 0.0 | | | | |
| 18B002A256 | 10 | 73.7 | 65-77 | 0.0 | | | | |
| JACK | 20 | 73.7 | 63-82 | | 18 | 68.4 | 52-87 | |
| (Control) | | | | | | | | |

All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the list of the foregoing embodiments and the appended claims.

### REFERENCES

1. McMullen, M., Jones, R., and Gallenberg, D. 1997. Scab of wheat and barley: A re-emerging disease of devastating impact. Plant Dis. 81:1340-1348
2. Smith & Waterman, 1981, Adv. Appl. Math. 2: 482
3. Needleman & Wunsch, 1970, J. Mol. Biol. 48: 443
4. Pearson & Lipman, 1988, Proc. Nat'l. Acad. Sci. 85: 2444
5. Altschul et al., 1990, J. Mol. Biol. 215: 403-410
6. Henikoff & Henikoff, 1989, Proc. Natl. Acad. Sci. 89: 10915
7. Karlin & Altschul, Proc. Nat'l. Acad. Sci. USA 90: 5873-5787 (1993)
8. Tijssen (1993) Laboratory Techniques in Biochemistry and Molecular Biology-Hybridization with Nucleic Acid Probes part I chapter 2, “Overview of principles of hybridization and the strategy of nucleic acid probe assays”, Elsevier, New York
9. Meinkoth and Wahl Anal. Biochem. 138:267-284 (1984)
10. Ausubel et al., eds. (1995) Current Protocols in Molecular Biology, Chapter 2 (Greene Publishing and Wiley-Interscience, New York).
11. Sambrook et al. (1989) Molecular Cloning: A Laborator Manual (2d ed., Cold Spring Harbor Laboratory Press, Plainview, N.Y.).
12. Bai, G.-H., and Shaner, G. 1994. Scab of wheat prospects for control. Plant Dis. 78:760-766.
13. Tuite, J., Shaner, G., and Everson, R. J. 1990. Wheat scab in soft red winter wheat in Indiana in 1986 and its relation to some quality measurements. Plant Dis. 74:959-962.
14. Martin, R. A., and Johnston, H. W. 1982. Effects and control of Fusarium diseases of cereal grains in the Atlantic Provinces. Can. J, Plant. Pathol. 4:210-216.
15. Broders, K. D., Lipps, P. E., Paul, P. A., Dorrance, A. E. 2007. Evaluation of Fusarium granimearum Associated with Corn and Soybean Seed and Seedling Disease in Ohio. Plant Disease. 91(9):1155-1160.
16. Canadian Seed Trade Association, Fusarium graminearum—The Corn Seed Perspective, March 2003. http://www.cdnseed.org/pdfs/press/Fusarium%20FactSheet.pdf
17. Dong, Z., Han, M., Feroroff, N. The RNA-binding proteins HYL1 and SE promote accurate in vitro processing of pri-miRNA by DCL1. PNAS. 105(29):9970-9975.
18. Subramanian S, Fu Y, Sunkar R, Barbazuk W B, Zhu J K, Yu O. Novel and nodulation-regulated microRNAs in soybean roots. BMC Genomics. 9:160 (2008).
19. Bartel (2004) Cell, 116:281-297
20. Allen et al. (2005) Cell, 121:207-221
21. Kim (2005) Nature Rev. Mol. Cell Biol., 6:376-385
22. Aukerman. and Sakai (2003) Plant Cell, 15:2730-2741
23. Parizotto et al. (2004) Genes Dev., 18:2237-2242
24. Lee et al. (2002) EMBO Journal, 21:4663-4670
25. Reinhart et al. (2002) Genes & Dev., 16:1616-1626
26. Lund et al. (2004) Science, 303:95-98
27. Millar and Waterhouse (2005) Funct. Integr Genomics, 5:129-135
28. Xie et al. (2004) PLoS Biol., 2:642-652
29. Bartel (2004) Cell, 116:281-297
30. Murchison and Hannon (2004) Curr. Opin. Cell Biol., 16:223-229
31. Dugas and Bartel (2004) Curr. Opin. Plant Biol., 7:512-520
32. Tomari et al. (2005) Curr. Biol., 15:R61-64
33. G. Tang (2005) Trends Biochem. Sci., 30:106-14
34. Kim (2005) Nature Rev. Mol. Cell Biol., 6:376-385
35. O'Donnell et al. (2005) Nature, 435:839-843
36. Cai et al. (2005) Proc. Natl. Acad. Sci. USA, 102:5570-5575
37. Morris and McManus (2005) Sci. STKE, pe41 (available online at stke.sciencemag.org/cgi/reprint/sigtrans; 2005/297/pe41.pdf
38. Ambros et al. (2003) RNA, 9:277-279
39. Xie et al. (2005) Plant Physiol., 138:2145-2154
40. Jones-Rhoades and Bartel (2004) Mol. Cell, 14:787-799
41. Sunkar and Zhu (2004) Plant Cell, 16:2001-2019
42. Griffiths-Jones et al. (2003) Nucleic Acids Res., 31:439-441
43. Kidner and Martienssen (2004) Nature, 428:81-84
44. Millar and Gubler (2005) Plant Cell, 17:705-721
45. Palatnik et al. (2003) Nature, 425:257-263
46. Rhoades et al. (2002) Cell, 110:513-520
47. Allen et al. (2004) Nat. Genet., 36:1282-1290
48. Dabney, S. M., J. D. Schreiber, C. S. Rothrock, and J. R. Johnson. 1996. Cover crops affect sorghum seedling growth. Agron. J 88:961-970.
49. Michael Zuker. Mfold web server for nucleic acid folding and hybridization prediction. Nucleic Acids Res. 31 (13), 3406-3415, 2003. http://mfold.rna.albany.edu/?q=mfold/RNA-Folding-Form

What is claimed is:

1-40. (canceled)

41. A method of expressing a anti-fungal miRNA in a plant, the method comprising;
a) providing a plant expression cassette consisting of at least two miRNA passenger sequences and at least two respective miRNA guide sequences operably linked to a promoter and terminator sequence wherein, the miRNA passenger sequences are selected from the group consisting of SEQ ID NOs: 1, 3, 5, and 7 and the guide sequences are selected from the group consisting of SEQ ID NOs: 2, 4, 6, or 8;
b) inserting the expression cassette of a) into the genome of a plant cell; and
c) generating a plant from the plant cell of b), wherein the plant expresses said anti-fungal miRNA.

42. The method of claim 41, wherein the miRNA passenger sequences are derived from Fusarium graminearum or Phakopsora pachyrhizi.

43. The method of claim 41, wherein the promoter is a constitutive promoter.

44. The method of claim 43, wherein the promoter is a Cestrum viral promoter.

45. The method of claim 41, wherein the terminator is a NOS terminator.

46. The method of claim 41, wherein the expressed miRNA reduces Phakopsora pachyrhizi spore germination by at least 41, 48, 81, 89, 93 or 94% as compared to a control plant.

47. A plant expression cassette comprising at least two miRNA passenger sequences and respective mi RNA guide sequences operably linked to a promoter and terminator sequence wherein, the mi RNA passenger sequences are selected from the group consisting of SEQ ID NOs: 1, 3, 5, and 7 and the guide sequences are selected from the group consisting of SEQ ID NOs: 2, 4, 6, and 8.

48. The plant expression cassette of claim 47, wherein the promoter is a Cestrum viral promoter and the terminator is a NOS terminator.

49. The plant expression cassette of claim 47, wherein the miRNA passenger sequences are derived from Fusarium graminearum or Phakopsora pachyrhizi.

50. A plant cell comprising the expression cassette of claim 47.

51. A plant comprising the expression cassette of claim 47, wherein said plant has increased resistance to a fungal pathogen as compared to a control plant.

52. A method of creating a plant having increased resistance to a fungal pathogen, the method comprising;
a) inserting into a plant cell an expression cassette consisting of at least two miRNA passenger sequences and respective miRNA guide sequences operably linked to a promoter and terminator sequence wherein, the miRNA passenger sequences are selected from the group consisting of SEQ ID NOs: 1, 3, 5, and 7 and the guide sequences are selected from the group consisting of SEQ ID NOs: 2, 4, 6, or 8; and
b) generating a plant from the plant cell of a) wherein said plant has increased resistance to a fungal pathogen as compared to a control plant.

53. The method of claim 52 wherein the plant is either maize or soy.

54. The method of claim 52 wherein the plant has increased resistance to either a Fusarium fungus or Phakopsora fungus.

55. A isolated single-stranded nucleic acid molecule comprising a fungal rRNA first sequence selected from the group consisting of SEQ ID NOs: 1, 3, 5 and 7 and a second sequence comprising a sequence capable of forming a duplex with said first sequence.

56. The isolated single-stranded nucleic acid molecule of claim 55, wherein the second sequence comprises a nucleic acid sequence selected from the group consisting of SEQ ID NOs: 2, 4, 6, and 8.
